(19) United States
(12) Patent Application Publication
(10) Pub. No.: US 2012/0226251 A1
(43) Pub. Date: Sep. 6, 2012

(54) FOAM DEVICE FOR LIQUID ABSORPTION AND METHOD

(75) Inventors: Daniel Rivest, Montreal (CA); Anja White, Oakdale, MN (US)

(73) Assignee: BIODAPTIVE, LLC, St. Paul, MN (US)

(21) Appl. No.: 13/150,862

(22) Filed: Jun. 1, 2011

Related U.S. Application Data
(60) Provisional application No. 61/448,025, filed on Mar. 1, 2011.

(51) Int. Cl. A61L 15/22; B23P 17/00

(52) U.S. Cl. 604/369; 29/527.1

(57) ABSTRACT

One aspect is a liquid absorption including a foam frame defining a cavity region and having a perimeter defining an area in a transverse direction. An insert is substantially contained within the cavity region. The cavity region has a perimeter defining an area in the transverse direction, the cavity area being the majority of the foam frame area. The foam frame and insert are configured to absorb liquid into the insert when the foam frame is placed adjacent a liquid source.

### CROSS REFERENCE TO RELATED APPLICATIONS

This Non-Provisional Patent Application claims the benefit of the filing dates of U.S. Provisional Patent Application Ser. No. 61/448,025, filed Mar. 1, 2011, entitled “FOAM DEVICE FOR LIQUID ABSORPTION AND METHOD,” which is herein incorporated by reference.

### BACKGROUND

One aspect relates to an absorbent foam device with a cavity configured to absorb and hold liquid and method of making and using the same. In certain applications it is useful to have an absorbent device to draw liquid away from an area to which the device is applied. For example, in wound care it is useful to place a foam device over a wound to draw fluid away from the wound. In some instances it is helpful if the fluid is not only drawn away from the wound, but also helpful if the fluid is not allowed to return to the wound. For these and other reasons, there is a need for the present invention.

### BRIEF DESCRIPTION OF THE DRAWINGS

The accompanying drawings are included to provide a further understanding of the present invention and are incorporated in and constitute a part of this specification. The drawings illustrate the embodiments of the present invention and together with the description serve to explain the principles of the invention. Other embodiments of the present invention and many of the intended advantages of the present invention will be readily appreciated as they become better understood by reference to the following detailed description. The elements of the drawings are not necessarily to scale relative to each other. Like reference numerals designate corresponding similar parts.

FIG. 1 illustrates an absorption device in accordance with one embodiment in a perspective view.

FIG. 2a illustrates an absorption device in accordance with one embodiment in a top view.

FIG. 2b illustrates an absorption device in accordance with one embodiment in a cross-sectional view.

FIG. 2c illustrates an absorption device in accordance with one embodiment in a detailed cross-sectional view.

FIGS. 3a-3c illustrate a process of forming an absorption device in accordance with one embodiment.

FIG. 4 illustrates an absorption device in accordance with one embodiment in a perspective view.

FIG. 5a illustrates an absorption device in accordance with one embodiment in a top view.

FIG. 5b illustrates an absorption device in accordance with one embodiment in a cross-sectional view.

FIG. 5c illustrates an absorption device in accordance with one embodiment in a detailed cross-sectional view.

FIGS. 6a-6d illustrate sectional views of foam from various portions of an absorption device in accordance with one embodiment.

FIG. 7a illustrates a frame for an absorption device in accordance with one embodiment.

FIG. 7b illustrates an absorption device in accordance with one embodiment.

FIG. 7c illustrates a frame surrounding an absorption device in accordance with one embodiment.

FIG. 8 illustrates an absorption device in accordance with one embodiment in a cross-sectional view.

### DETAILED DESCRIPTION

In the following Detailed Description, reference is made to the accompanying drawings, which form a part hereof, and in which is shown by way of illustration specific embodiments in which the invention may be practiced. In this regard, directional terminology, such as “top,” “bottom,” “front,” “back,” “leading,” “trailing,” etc., is used with reference to the orientation of the Figure(s) being described. Because components of embodiments of the present invention can be positioned in a number of different orientations, the directional terminology is used for purposes of illustration and is in no way limiting. It is to be understood that other embodiments may be utilized and structural or logical changes may be made without departing from the scope of the present invention. The following detailed description, therefore, is not to be taken in a limiting sense, and the scope of the present invention is defined by the appended claims.

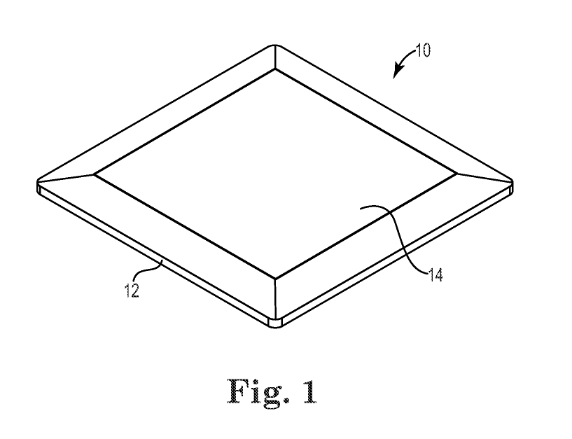

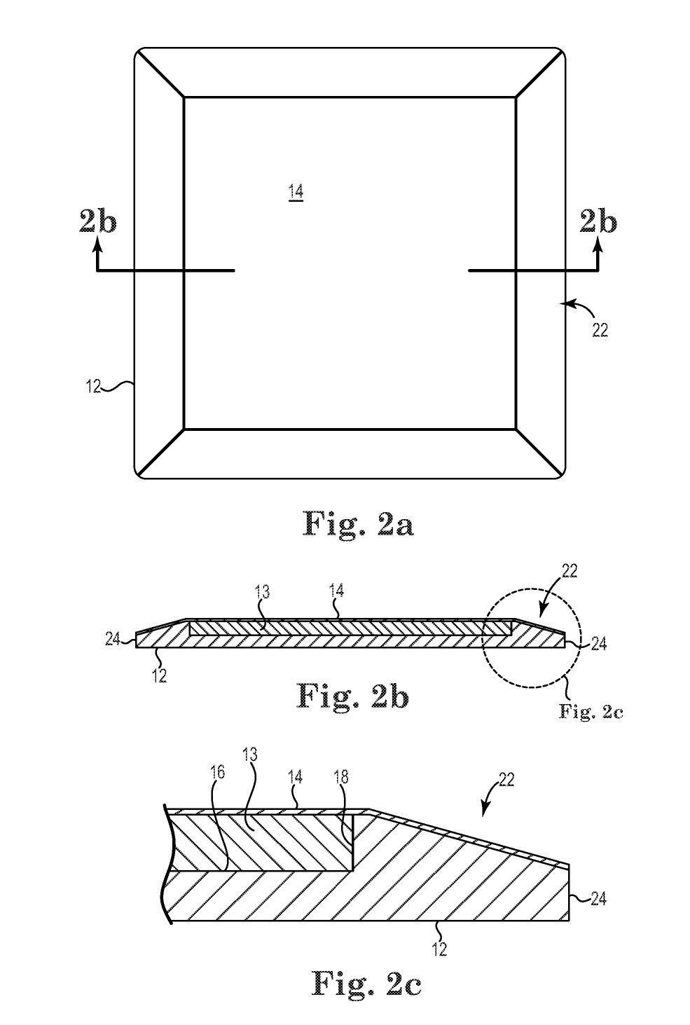

FIG. 1 illustrates absorption device 10 according to one embodiment. Absorption device 10 includes frame 12, insert 13 (illustrated in FIGS. 2b-2c) and cover 14. In one embodiment, frame 12 includes a cavity into which insert 13 is placed. Cover 14 is then placed over the combination of frame 12 and insert 13 to substantially contain insert 13 within frame 12 and cover 14. In one embodiment, absorption device 10 is configured to efficiently and effectively absorb liquid up in to the device 10 from a surface over which it is placed. In one example application, absorption device 10 can be placed over a wound and effectively absorb blood, exudates, and other fluids up into insert 13 and away from the wound. In one embodiment, liquid is then retained within absorption device 10, thereby substantially preventing the absorbed liquid from returning to the wound.

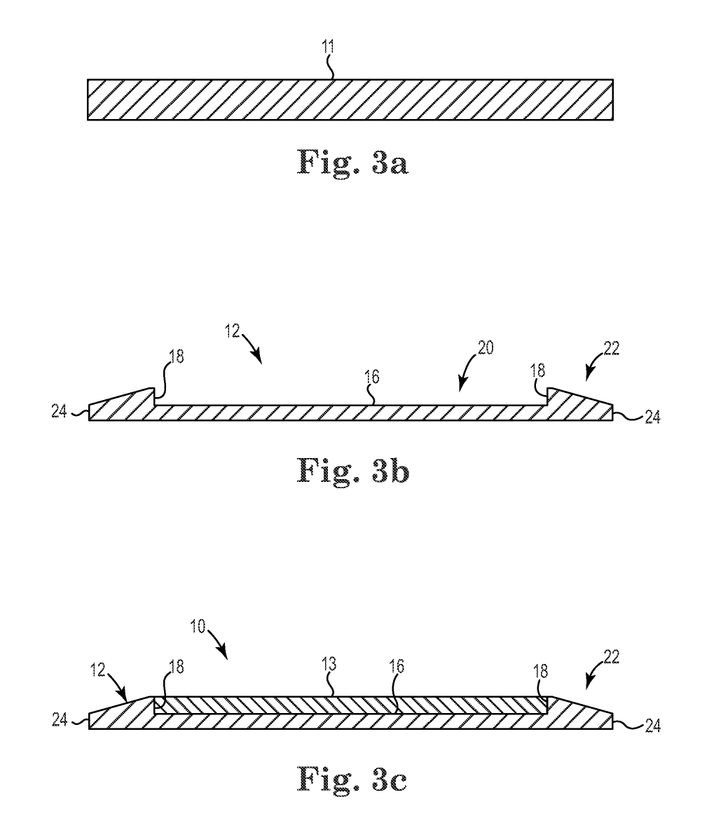

FIG. 2a illustrates a top view of absorption device 10 in accordance with one embodiment. FIG. 2b illustrates a cross-section view of absorption device 10 taken along line 2b-2b in FIG. 2a. As illustrated, frame 12 defines a cavity (labeled as cavity 20 in FIG. 3b) into which insert 13 is placed. In one embodiment, insert 13 is configured of a material that provides excellent transfer of liquid, such that liquid is readily drawn up through frame 12 and into insert 13. In one example, a superabsorbent paper can be used for insert 13.

FIG. 2c illustrates a further detail of the cross-sectional view of absorption device 10 from FIG. 2b in the area labeled 2c. In the illustration, cavity edge 18 and cavity floor 16 of frame 12 define the cavity into which insert 13 is seated. In one embodiment, cavity edge 18 is substantially vertical and cavity floor 16 is substantially horizontal, as oriented in the illustration. In one embodiment, insert 13 has an outer periphery that substantially matches the shape of the cavity defined by cavity edge 18, such that insert 13 fits snuggly in the cavity of frame 12.

In one embodiment, cover 14 is a polyurethane film that is placed over the combination of frame 12 and insert 13 to substantially contain insert 13 within the cavity of frame 12. In one embodiment, cover 14 allows moisture to evaporate through it, yet it provides an effective barrier to prevent contaminants from entering absorption device 10 and insert 13. Where absorption device 10 is used in a wound-care application, cover 14 allows some moisture to evaporate from the wound, and it provides an effective barrier to prevent contaminants from entering the wound.

In one embodiment, frame 12 of absorption device 10 is a foam material. In one embodiment, the foam material 12 provides good transfer of liquid. In one embodiment, the foam of frame 12 readily wicks liquid away from a surface over which it is placed and provides it to insert 13 where the liquid is readily absorbed.

In one embodiment, frame 12 is a foam body that is thermoformed into its shape. FIGS. 3a-3c illustrate the forming and assembly of absorption device 10 in accordance with one embodiment. In one example, frame 12 is an open-cell polyurethane foam that is thermoformed into its shape, including the shape of its cavity. FIG. 3a illustrates a foam piece 11 before a thermoforming process. FIG. 3b illustrates frame 12, which is produced by thermoforming foam piece 11 into the illustrated shape.

The thermoforming process can be any of a number of known techniques for using heat to form a foam piece 11 into a desired shape. For example, a mold cavity can be formed by bringing two mold halves together, where the mold cavity has an interior shape complementary to the shape of frame 12 in FIG. 3b. As such, introducing foam piece 11 into such a cavity, closing the mold, and then heating can form the desired shape for frame 12 from inserted foam piece 11.

As illustrated in FIG. 3b, frame 12 includes cavity floor 16, cavity edge 18, cavity 20, device edge regions 22 and ends 24. In one embodiment, cavity floor 16 and cavity edges 18 define cavity 20 into which insert 13 will be placed. In one embodiment, the thermoforming process also produces device edge regions 22. In one embodiment, the thickness of frame 12 in device edge regions 22 gradually decreases from each of cavity edges 18 out toward the respective ends 24 of device 10. This “beveled” shape at device edge regions 22 of frame 12 can be useful in certain applications. For example, the beveled device edge regions 22 can reduce friction between absorption device 10 and the surface against which device 10 is placed. For example, where absorbent device 10 is used in a wound-care application and is placed over a wound, the beveled device edge regions 22 of frame 12 help it lay flat, making any taping of the device easier.

Because frame 12 begins as a substantially rectangular shaped foam piece 11 in one embodiment, when it is thermoformed into the shape illustrated in FIG. 3b, certain areas of frame 12 are have a higher density of foam than other areas. For example, the area of frame 12 that is directly below cavity floor 16 has a higher foam density than does the area just outside cavity walls 18. Foam density then increases again moving further out toward end 24 due to the beveled shape of device edge regions 22.

FIG. 3c illustrates insert 13 seated into cavity 20 of frame 12 before cover 14 is added. In one embodiment, insert 13 is an absorbent material. Because insert 13 is seated in cavity 20 and not restrained above it, even after cover 14 is added, insert 13 can expand upwards, or away from cavity floor 16, as it absorbs additional liquid through frame 12. As it expands, cover 14 simply moves up with it. As such, insert 13 is configured to expand within cavity 20 and can absorb and hold a large amount of liquid.

In one embodiment, the absorbent substrate of insert 13 is not attached to the surfaces 16 and 18 of cavity 20 with an adhesive, but is rather simply seated within cavity 20. Once cover 14 is added, cavity 20 is essentially a container for insert 13. As such, the material of insert 13 is essentially “free floating.” In one embodiment, the moisture vapor transmission rate (MVTR) of absorption device 10 is increased from it would be if a layer of adhesive was used between cavity floor 16 and insert 13 or between insert 13 and cover 14.

In one embodiment where absorbent device 10 is used in a wound-care application, insert 13 also includes materials that will help solidify liquids that are absorbed into it. In such an example, liquid that is drawn into absorbent device 10 is pulled into insert 13 where it is then at least partially solidified and held therein. Solidifying the liquid absorbed makes it more unlikely for any liquid to return back through frame 12 and back to the wound over which absorbent device 10 is placed. This can be important for avoiding contamination and decreasing the likelihood of wound infection.

In other embodiments of absorption device 10, cavity 20 of frame 12 can be filled with a variety of inserts to accomplish other functions. For example, in one embodiment, an insert 13 can, in addition to absorb and hold liquid, also or alternatively be configured to hold active ingredients that are released back through frame 12 to the source of the liquid. For example, in a wound-care application, healing agents may be suspended in insert 13 and release back down to the wound as liquid from the wound is absorbed into insert 13, thereby aiding in the healing process. At the same time, liquid can be prevented from returning back through frame 12 to the wound by partially solidify the liquid as it enters insert 13.

The thermoprocessing of a foam piece 11 to form frame 12 can be used to create an absorbent device 10 of a large variety of shapes and sizes in accordance with various embodiments. For example, although absorbent device 10 has been illustrated with a generally square-shaped periphery, it could readily be made into a circular, rectangular or any variety of custom shapes desired for particular applications. In addition, the size can be readily adjusted for the application as well.

Furthermore, although cavity 20 has been illustrated with a generally square-shaped periphery, it could readily be made into a circular, rectangular or any variety of custom shapes desired for particular applications. Insert 13 could then correspondingly be shaped to match the selected shape of cavity 20. In addition, the relative size of cavity 20 to device edge region 22 can be readily adjusted. For example, the relative size of cavity 20 and insert 13 is larger (wider, for example, in the cross-section illustrated in FIG. 2b) than is the size of device edge regions 22. In some embodiments, the size of device edge regions 22 can be widened while the size of cavity 20 and insert 13 can be narrowed.

In one embodiment, the relative size of cavity 20 allows for a relatively large insert to be used to absorb liquid. For example, where absorption device 10 is used in a wound-care application to absorb blood, exudates, and other fluids up into insert 13 and away from the wound, a relatively large and absorbent insert 13 can be used. In one case, cavity 20 has a relatively large area that occupies a substantial amount of the total area of frame 12, thereby allowing for an insert 13 that effectively absorbs liquid. For example, in a direction transverse to the surface over which absorbent device 10 would be placed, cavity edges 18 define a perimeter of the cavity 20 that has a cavity area in the transverse plane. Similarly, the perimeter of frame 12 at ends 24 also defines a foam frame area in the transverse plane. In one embodiment, the cavity area of cavity 20 is larger than the remaining foam frame area of frame 12, which is outside cavity 20.

In one embodiment, the area of cavity 20 is at least 50% of the total area of frame 12. For example, in the cross-section illustrated in FIG. 2b, if the distance between ends 24 is 4 inches, the distance between cavity edges 18 would be at least 2 inches. Although the illustration gives a relatively square configuration, similar relative areas for cavity 20 and frame 12 can be used in circular, rectangular and other shaped absorbent device 10.

In addition, other techniques can be used to form frame 12 and cavity 20 in accordance with other embodiment. For example, in other embodiments, the shape of frame 12 and cavity 20 could be formed using pressure and/or by grinding the material into the desired shapes.

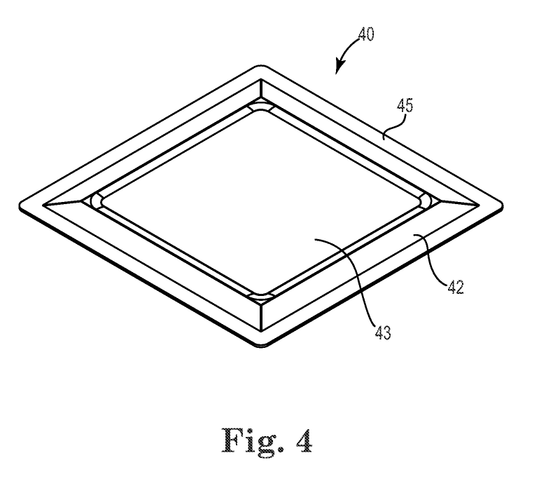

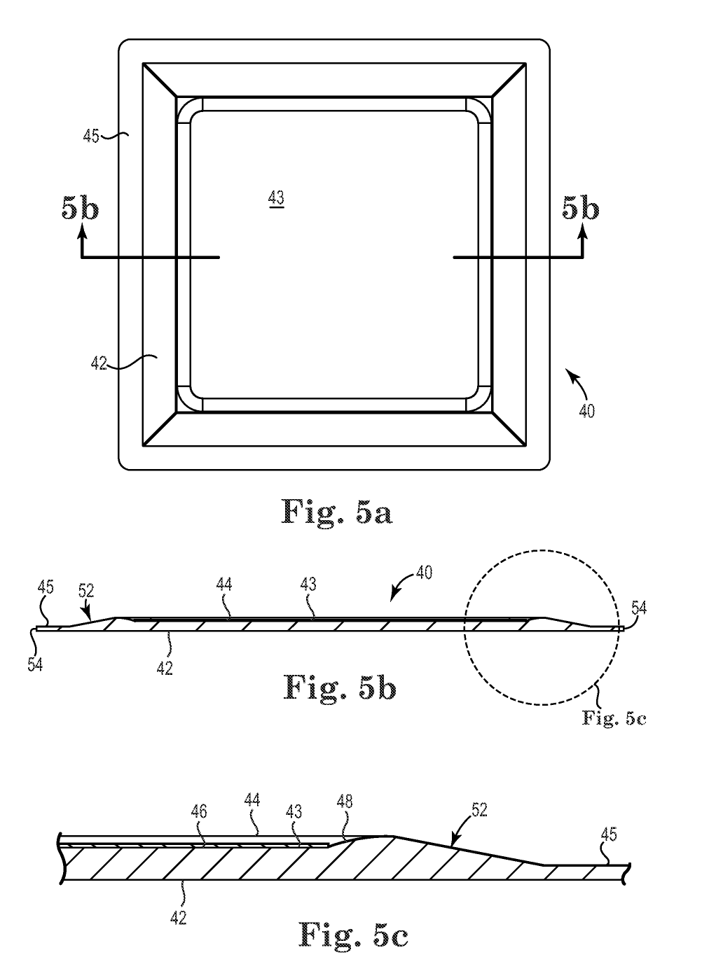

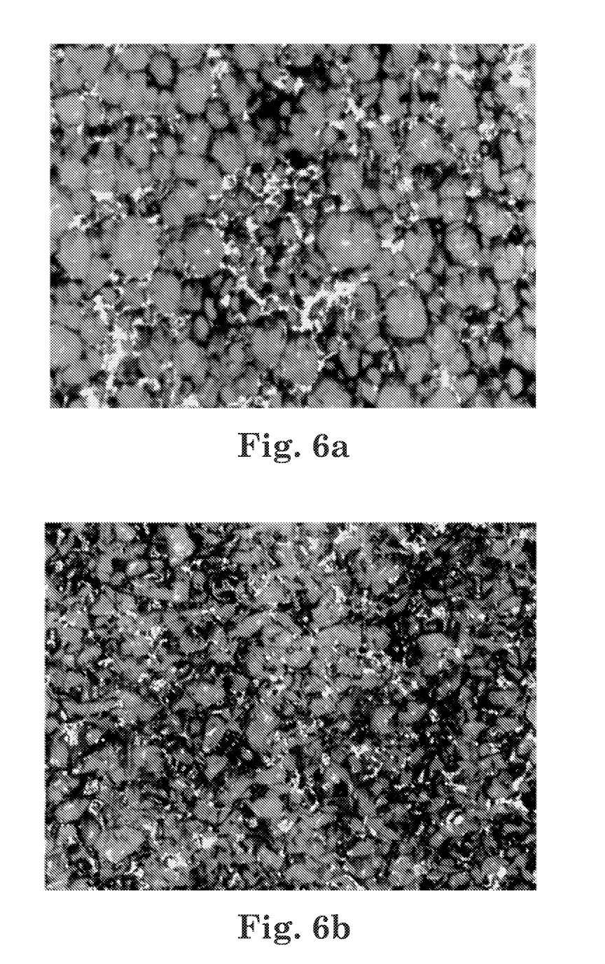

FIG. 4 illustrates absorption device 40 according to one embodiment. Absorption device 40 includes frame 42, insert 43, cover 44 (illustrated in FIGS. 5b-5c, but left off in FIGS. 4 and 5a to show insert 43) and lip 45. In one embodiment, frame 42 includes a cavity into which insert 43 is placed and over which cover 44 is placed (FIGS. 5b-5c). As with device 10 above, in one embodiment, absorption device 40 is configured to efficiently and effectively absorb liquid up into insert 43 of device 40 from a surface over which it is placed.

In the illustration of FIG. 5c, it is evident that the height of insert 43 is less than the overall height of the cavity of frame 42. As such, there is space between the top of insert 43 and cover 44 in one embodiment. In other embodiments, the height of the insert may be substantially equal to the height of the cavity (such as illustrated in FIG. 2c), or it may be even greater than the height of the cavity.

In addition to some of the features described above with respect to device 10, absorbent device 40 also includes lip 45, which in one example, surrounds the periphery of the device. In one example application, absorption device 40 can be placed over a wound and effectively absorb blood, exudates and other fluids up into the device and away from the wound. In one embodiment, lip 45 also effectively creates an occlusive dressing that can be an air and/or water-tight trauma dressing.

As with device 10 above, frame 42 of device 40 is configured with a cavity that can be filled with substrates or inserts. Frame 42 can be thermoformed with a cavity that functions as “container” for a layer or layers to be inserted therein, with cover 44 acting as the top. In one embodiment, frame 42 is made of a foam material that provides a good transfer of liquid from a surface over which device 40 is placed up and into insert 43. Although devices 10 and 40 are illustrated with single inserts 13 and 43, other embodiments can include more insert layers in the cavity, as desired.

FIG. 5a illustrates a top view of absorption device 40 in accordance with one embodiment (with cover 44 left off for illustration). FIG. 5b illustrates a cross-section view of absorption device 40 taken along line 5b-5b in FIG. 5a. As illustrated, frame 42 defines a cavity into which insert 43 is placed. In one embodiment, insert 43 is configured of an absorbent material such that liquid is readily drawn up through frame 42 and into insert 43. As with device 10 described above, various embodiments of insert 43, or other additional inserts, are designed to hold and solidify liquids or to hold active ingredients to be released, into a wound for healing purposes, for example.

FIG. 5c illustrates a further detail of the cross-sectional view of absorption device 40 from FIG. 5b in the area labeled 5c. In the illustration, cavity edge 48 and cavity floor 46 of frame 42 define the cavity into which insert 43 is seated. In one embodiment, cavity edge 48 is not substantially vertical (as cavity edge 18 was illustrated and described above), but has a more sloped edge. In one embodiment, insert 43 generally has an outer periphery that substantially matches the shape of the cavity defined by cavity edge 48, such that insert 43 fits snuggly in the cavity of frame 42. The cavity formed by raised cavity edge 48 is essentially a container, bounded at the top by cover 44, for holding insert 43.

As with device 10 above, in one embodiment frame 42 of absorption device 40 is a foam material, such as an open cell polyurethane foam that is thermoformed into its shape, including the shape of its cavity, its beveled edges, and lip 45. The thermoforming process can be any of a number of known techniques for using heat to form a foam piece into a desired shape, as described above relative to device 10.

As illustrated in FIGS. 5b-5c, frame 42 includes cavity floor 46, cavity edge 48, device edge regions 52 and ends 54. In one embodiment, cavity floor 46 and cavity edges 48 define a cavity into which insert 43 is placed. In one embodiment, the thermoforming process also produces device edge regions 52, which are beveled shaped, such that frame 42 gradually decreases in thickness from each of cavity edges 48 out toward the respective lips 45, which can also be formed in the thermoforming process. As above, these beveled edges can reduce friction between absorption device 40 and the surface against which device 10 is placed.

Many of the above-described features of device 10 equally apply to device 40. For example, insert 43 is seated into the cavity of frame 42 and is a superabsorbent paper that is an excellent absorbent of liquids. Also, in one embodiment, insert 43 is not attached to the surfaces of the cavity of frame 42 so that it is free floating and has a good MVTR. Also, where absorbent device 40 is used in a wound-care application, insert 43 includes materials that will help solidify liquids that are absorbed into it to prevent liquid from returning back to the wound. It can also contain healing agents for release. Cover 44 is a polyurethane film that allows moisture evaporation away from the wound and prevents contamination from reaching the wound.

As with device 10, the thermoprocessing of a foam piece to form frame 42 can be used to create an absorbent device 40 of a large variety of shapes and sizes in accordance with various embodiments. Because frame 42 begins as a substantially rectangular shaped foam piece in one embodiment, when it is thermoformed into the shape illustrated in FIG. 5b, certain areas of frame 42 are have a higher density of foam than other areas.

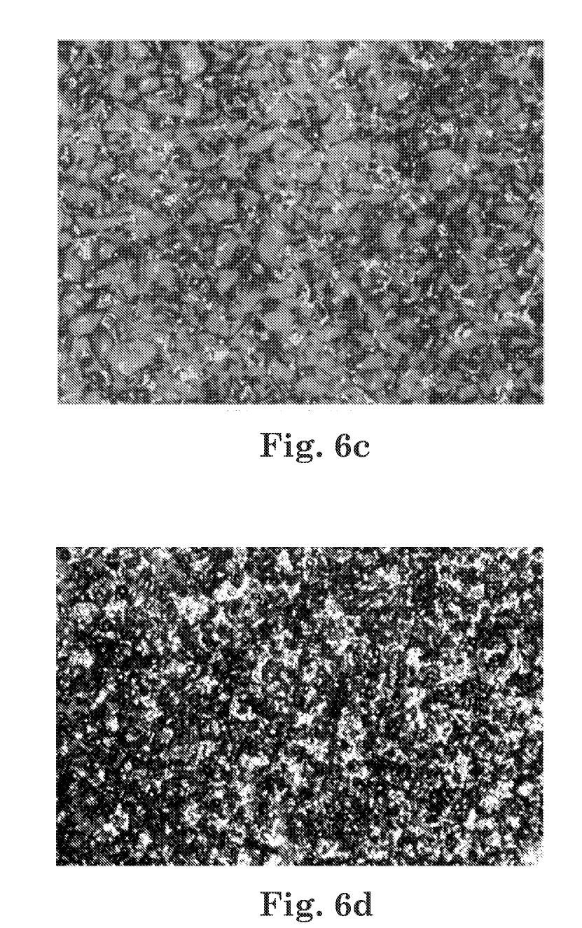

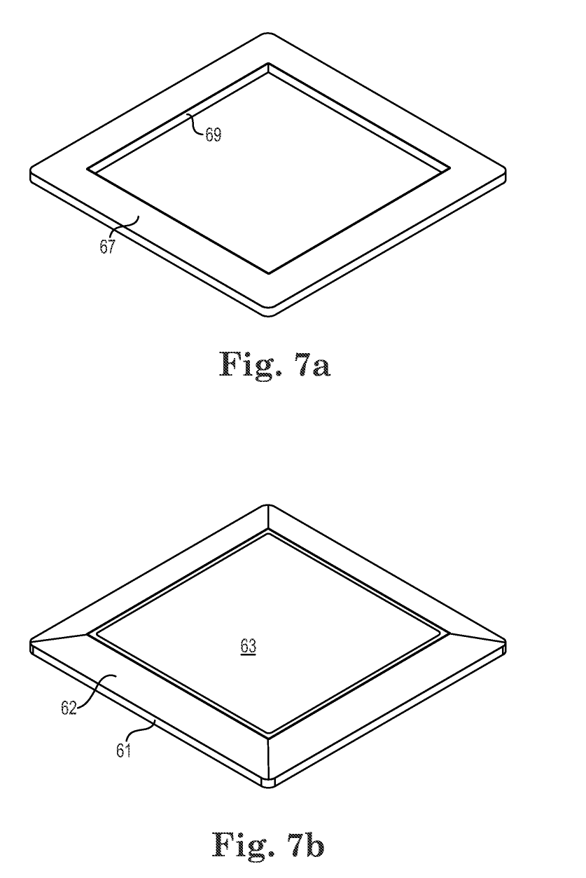

FIGS. 6a-6d illustrate this variation in foam density with sectional views of foam from various portions of an absorption device in accordance with one embodiment. FIG. 6a illustrates foam that has not been thermoprocessed, such as foam block 11 in FIG. 3a. As is evident, the cells are relatively large and the foam material is therefore quite absorbent and transfer liquid quite readily.

FIG. 6b illustrates foam in the center portion of frame 42 directly below cavity floor 46. This area has higher density due to the thermoprocessing that created the cavity of frame 42. As is evident, the cells are relatively smaller than where no thermoprocessing had occurred, such as in FIG. 6a. The foam material is still absorbent in this area and transfers liquid quite readily.

FIG. 6c illustrates foam in device edge regions 52, just outside the cavity of frame 42. This area has a lower relative density, because, even though it is thermoprocessed to create the beveling, frame 42 is thicker in this region than in the cavity. As such, the cells in the foam of device edge regions 52 are larger relative to those in the area of the cavity seen in FIG. 6b, and foam material is absorbent in this area and will transfer liquid.

Finally, FIG. 6d illustrates foam in lip 45 near the edge 54. This area has a very high relative density, because it is thermoprocessed to create lip 45 and frame 42 is at it thinnest in this region. As such, the cells in the foam of lip 45 are quite small relative to all the other areas and here the foam could create a zone that is non-absorbent and will not transfer liquid.

In one embodiment, an adhesive may be added to the underside of the lip 45. In some instances, it can be difficult to effectively apply an adhesive to polyurethane foam, which is typically quite absorbent. Where the foam has been thermopressed into a high density state, such as in lip 45, such adhesives can be more effectively applied. As such, in certain wound care applications, lip 45 with an applied adhesive, can form an adhesive gasket around the wound, while supplying an effective absorption device in insert 43.

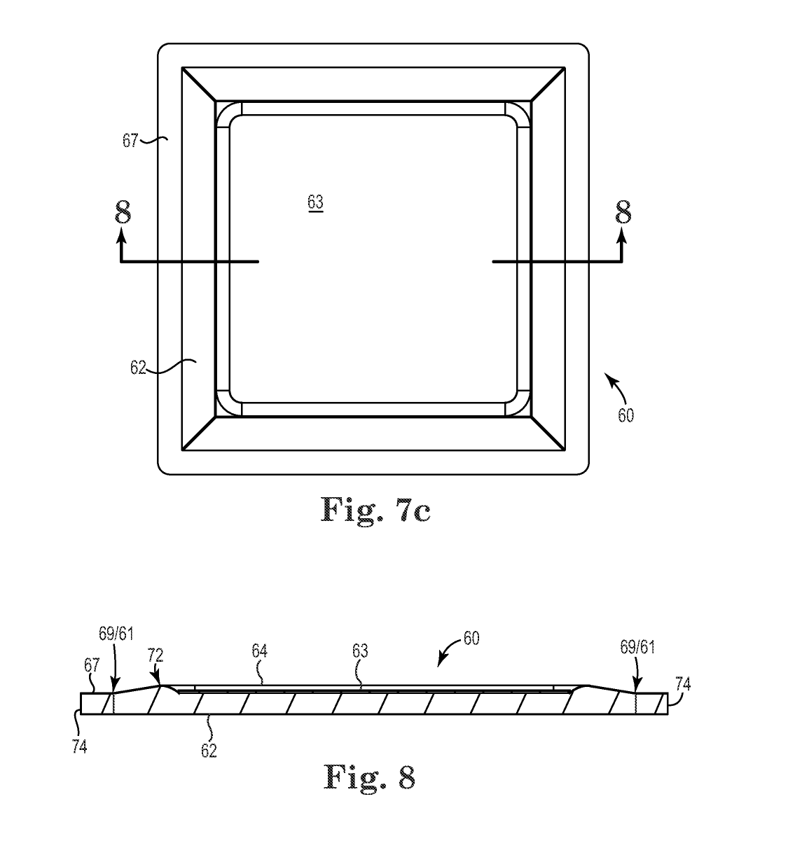

FIGS. 7a-7c and 8 illustrate absorption device 60 according to one embodiment. Absorption device 60 includes frame 62, insert 63, cover 64 (illustrated in FIG. 8, but left off in FIGS. 7a-7c to show insert parts below) and border 67. In one embodiment, border 67 is a ring-like shape, illustrated in FIG. 7a, into which frame 62, illustrated in FIG. 7b, is inserted. In one embodiment, border 67 has an inner perimeter surface 69 that substantially matches the outer perimeter surface 61 of frame 62 such that there is a snug fit between them.

As illustrated in the sectional view of FIG. 8, once frame 62 is placed within border 67, cover 64 is placed over the combination, and adhered to both, thereby coupling them together. In one embodiment, frame 62 is similar to frame 42 above and includes a cavity, with a cavity floor and cavity edges, into which insert 63 is placed and over which cover 64 is placed. In one embodiment, frame 62 of absorption device 60 is similar to frame 42 above and includes device edge regions 72 and ends 74. In one embodiment, cover 64 is adhered to device edge regions 72 and a top surface of border 67. Since the cavity floor is recessed relative to the edges of the cavity, cover 64 is extends above the cavity defining the space into which insert 63 is placed.

As with device 40 above, in one embodiment, absorption device 60 is configured to efficiently and effectively absorb liquid up into insert 63 of device 60 from a surface over which it is placed. In one embodiment, border 67 is an adhesive border that is added around frame 62 to provide an adhesive bond between absorption device 60 and the surface over which it is placed. As discussed above, in some instances it can be difficult to effectively apply an adhesive to polyurethane foam, such as used in some embodiments for frame 62. Accordingly, in one embodiment, border 67 is a polyurethane material that can readily carry an adhesive on its lower surface such that it readily adheres to a surface over which it is placed.

Although specific embodiments have been illustrated and described herein, it will be appreciated by those of ordinary skill in the art that a variety of alternate and/or equivalent implementations may be substituted for the specific embodiments shown and described without departing from the scope of the present invention. This application is intended to cover any adaptations or variations of the specific embodiments discussed herein. Therefore, it is intended that this invention be limited only by the claims and the equivalents thereof.

What is claimed is:

1. A liquid absorption device comprising:
a foam frame defining a cavity region and having a perimeter defining a foam frame area in a transverse direction; and
an insert substantially contained within the cavity region;
wherein the cavity region has a perimeter defining a cavity area in the transverse direction, the cavity area occupying the majority of the foam frame area;
wherein the foam frame and insert are configured to absorb liquid into the insert when the foam frame is placed adjacent a liquid source.

2. The liquid absorption device of claim 1, wherein the foam frame comprises cavity walls forming a periphery that substantially contains the cavity, the cavity walls being proximate to edges of the liquid absorption device.

3. The liquid absorption device of claim 1, wherein the cavity region of the frame substantially contains the insert.

4. The liquid absorption device of claim 3, characterized in that the absorption pad not adhered to the frame with adhesive

5. The liquid absorption device of claim 1, wherein the insert is an absorbent insert pad, and wherein the cavity region is configured to accommodate expansion of the insert pad as it absorbs liquid.

6. The liquid absorption device of claim 5, wherein absorbent insert pad is a superabsorbent paper.

7. The liquid absorption device of claim 1, wherein the frame is characterized in having substantially beveled edges.

8. The liquid absorption device of claim 7, wherein the beveled edges are around substantially all of a periphery of the absorption device.

9. The liquid absorption device of claim 1, wherein the frame is characterized in having a lip substantially surrounding the frame.

10. The liquid absorption device of claim 9, wherein the lip comprises a condensed foam such that the lip is substantially non-absorbent.

11. The liquid absorption device of claim 10, further comprising an adhesive added to lip to form an adhesive gasket substantially circumferentially containing the cavity region.

12. A liquid absorption device comprising:
a foam frame defining a cavity region;
an insert substantially contained within the cavity region;
a border substantially surrounding the foam frame, the boarder comprising a polyurethane material that can readily carry an adhesive; and
a cover adhered over at least a portion of the foam frame and over the border such that the cover couples the foam frame to the border and such that the cover contains the insert within the cavity region.

13. The liquid absorption device of claim 12, wherein the foam frame has a perimeter defining a foam frame area in a transverse direction, wherein the cavity region has a perimeter defining a cavity area in the transverse direction, and wherein the cavity area occupies the majority of the foam frame area.

14. The liquid absorption device of claim 12, wherein the foam frame and insert are configured to absorb liquid into the insert when the foam frame is placed adjacent a liquid source.

15. The liquid absorption device of claim 12, wherein the foam frame comprises cavity walls forming a periphery that substantially contains the cavity, the cavity walls being proximate to edges of the liquid absorption device.

16. The liquid absorption device of claim 3, characterized in that the absorption pad not adhered to the frame with adhesive and in that the frame is characterized in having substantially beveled edges

17. The liquid absorption device of claim 12, wherein border comprises a substantially non-absorbent material.

18. A method of making a liquid absorption device comprising:
forming a foam frame such that it defines a cavity region, such that it has a perimeter defining a foam frame area in a transverse direction, such that the cavity region has a perimeter defining a cavity area in the transverse direction, and such that the cavity area occupies a majority of the foam frame area; and
placing an insert into the cavity region such that it is substantially contained therein and such that it readily absorbs liquid when the foam frame and insert are placed adjacent a liquid source.

19. The method of claim 18, wherein forming the foam frame comprises one of a group comprising a thermoforming process, a pressure forming process and grinding process.
